(19) United States
(12) Patent Application Publication
(10) Pub. No.: US 2025/0311729 A1
(43) Pub. Date: Oct. 9, 2025

(54) NITRIC OXIDE PRECURSORS AND MULTI-COMPONENT COMPOSITIONS FOR FORMING THE SAME

(71) Applicant: Sterile State, LLC, Farmington Hills, MI (US)

(72) Inventor: Megan Cecelia Frost, Hancock, MI (US)

(73) Assignee: Sterile State, LLC, Farmington Hills, MI (US)

(21) Appl. No.: 18/863,461

(22) PCT Filed: Apr. 28, 2023
(86) PCT No.: PCT/US2023/020331

§ 371 Date: Nov. 6, 2024

Related U.S. Application Data
(60) Provisional application No. 63/341,320, filed on May 12, 2022.

(51) Int. Cl. A01N 59/00; A01P 1/00

(52) U.S. Cl. CPC A01N 59/00; A01P 1/00

(57) ABSTRACT

A nitric oxide precursor for providing nitric oxide is provided herein. In especially contemplated aspects the nitric oxide precursor comprises a reaction product of a (preferably primary) thiol-containing compound and a nitrosating compound. The thiol-containing compound and the nitrosating compound are typically reacted in the presence of a solvent, and optionally a catalyst/reducing equivalent, to form the nitric oxide precursor, which will then rapidly decompose to form the nitric oxide.

### RELATED APPLICATIONS

This application claims priority to co-pending US Provisional patent application with the Ser. No. 63/341,320, which was filed May 12, 2022, and which is incorporated by reference herein.

### FIELD OF THE INVENTION

The present disclosure generally relates to compositions and methods for nitric oxide precursors that can release nitric oxide, and especially nitric oxide precursors that comprise a reaction product of a (preferably primary) thiol-containing compound and a nitrosating compound.

### BACKGROUND

A variety of products and articles, including, for example, medical instruments, devices, and equipment, must be sterilized prior to use to prevent bio-contamination of a wound site, a sample, an organism, or the like. Similarly, while many detergents provide some reduction in microbial count, most detergent compositions will not have a microbicidal component, which would be desirable in a number of uses. Moreover, while there are certain formulations are known in the art that mask malodorous byproducts of microbial metabolism, such formulations typically lack a microbicidal component.

A number of sterilization processes are used which involve contacting the product or article with a sterilant. Examples of such sterilants include dintitrogen tetraoxide, nitric oxide, steam, ethylene oxide, hydrogen peroxide, dry heat, and the like. Conventional methods for forming nitric oxide use catalytic and enzymatic generation of nitric oxide from nitrite or NO donating compounds such as diazeniumdiolates. Such conventional methods for forming nitric oxide typically require expensive reactants and must in most cases be utilized in controlled systems to permit safe operation and generation of nitric oxide. These and other drawbacks have prevented mainstream commercialization of sterilization or sanitation composition capable of generating nitric oxide by consumers and professionals.

More recently, nitric oxide releasing compositions and devices have been developed in which a tertiary nitrosothiol compound is covalently coupled to a polymer (SNAP-PDMS), and in which illumination of the tertiary nitrosothiol compound results in decomposition of the compound to so form nitric oxide as is described in WO 2022/164894. Additional polymer compounds that contain various nitrosothiols that release nitric oxide upon irradiation are described in U.S. Pat. No. 9,884,943 and WO 2020/018488. While such compounds and compositions provide various advantages in certain use cases, the release of nitric oxide is relatively slow and typically requires energy directed to the compound. Therefore, currently known compositions and methods fail to produce substantial quantities of nitric oxide in a bolus or on-demand.

Accordingly, there remains an opportunity for improved compositions capable of generating nitric oxide for a variety of medical and consumer purposes, particularly where the nitric oxide is rapidly produced in substantial quantities.

### BRIEF SUMMARY OF THE INVENTION

A nitric oxide precursor for providing nitric oxide is provided herein. The nitric oxide precursor comprises, consists essentially of, consists of, or is, a reaction product of a thiol-containing compound and a nitrosating compound. In particularly preferred aspects, the thiol is a primary thiol. The thiol-containing compound and the nitrosating compound are reacted in the presence of a solvent to form the nitric oxide precursor. The nitric oxide precursor is capable of decomposing to form the nitric oxide. Where desired, a catalyst/reducing equivalent may be added to enhance formation of the nitric oxide precursor. In various embodiments, the nitric oxide precursor comprises a (preferably primary) nitrosothiol that is capable of rapidly decomposing to form the nitric oxide. It is contemplated herein that the nitric oxide precursor is capable of decomposing to form the nitric oxide on-demand, when desired, based on when the thiol-containing compound and the nitrosating compound are reacted in the presence of the solvent. Viewed from a different perspective, contemplated nitric oxide precursors will have significant chemical instability such that formation of the nitric oxide precursor is immediately followed by its decomposition to nitric oxide. As described in greater detail below, the on-demand formation of the nitric oxide precursor is suitable for a variety of applications requiring such control for forming form the nitric oxide.

The nitric oxide precursor may be used in a wide variety of medical and consumer applications. The properties of the nitric oxide precursor may be adjusted based on the selection of the thiol-containing compound and the nitrosating compound to suit specific applications. Non-limiting examples of suitable adjustments include nitric oxide generation capabilities and the rate of release of nitric oxide. As described in greater detail below, the nitric oxide precursor may be formed in situ from a multi-component composition. This in situ formation of the nitric oxide precursor may also be adjusted to suit specific applications by tuning formation and stability properties of the nitric oxide precursor such that nitric oxide can be, for example, generated rapidly and in a controlled manner. This controlled nitric oxide release is useful for sterilizing and sanitizing medical and consumer devices.

In particular, the multi-component composition may be utilized for forming solutions that generate nitric oxide in a controlled and predicable manner from a matrix/solution phase. These compositions may comprise a first component comprising the thiol-containing compound and a second component comprising the nitrosating compound to form small molecule nitric oxide donors that can be used for formulations and applications relating to disinfecting, sanitizing, and sterilizing washes for a wide variety of objects and devices and in a number of applications.

In some embodiments, the multi-component composition comprises the first component comprising the thiol-containing compound and the second component comprising the nitrosating compound with the first component and the second component isolated from each other. At least one of the first component and the second component may comprise the solvent.

In other embodiments, the multi-component composition comprises the first component comprising the thiol-containing compound having a particle size of from about 1 nm to about 10 mm and the second component comprising the nitrosating compound having a particle size of from about 1 nm to about 10 mm.

In various embodiments, the thiol-containing compound comprises a cysteine or derivative thereof, a thiol-derivatized polymer or filler, or a combination thereof. In exemplary embodiments, the cysteine or derivative thereof comprises cysteine, glutathione, acetyl cysteine, penicillamine, acetylpenicillamine, S-nitroso-n-acetylpenicillamine, bucillamine, or combinations thereof. Preferably, the thiol-containing compound comprises a primary thiol group, however, secondary and tertiary thiols are also contemplated herein. In these and other embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnittrite, nitrite salts, ion paired nitrite, silver nitrite, zinc nitrite, iron nitrite, copper nitrite, transition metal-nitrite compounds, or combinations thereof. Additionally, and where desired, a reducing equivalent may be used to enhance the reaction between the thiol compound and the nitrosating compound, and especially contemplated reducing equivalents include transition metals, other thiols, NADH, ascorbic aids, etc.

The inventors contemplate that the nitric oxide precursor may exhibit decomposition to nitric oxide after forming the reaction product within a predetermined amount of time, such as within 1 hour or less. In various embodiments, decomposition to nitric oxide may be sustained for a predetermined amount of time, such as a time period of from about 1 second to 1 minute, or from about 1 minute to about 10 minutes, or from about 10 minutes to 1 hour, or from about 1 hour to 6 hours, or from about 6 hours to 24 hours, or from 1 day to about 10 days, and even longer. Without being bound by theory, it is believed that in situ formation of the reaction product provides controlled decomposition of the nitric oxide precursor (e.g., (primary) nitrosothiol) to nitric oxide within a predetermined amount of time.

Viewed from a different perspective, contemplated multi-component compositions may be formulated as a detergent composition or a cleaning composition. The detergent or cleaning composition may be formulated as a liquid, powder, single-phase or multi-phase unit dose, pouch, tablet, gel, paste, bar, or flake. In these and other embodiments, the detergent or cleaning composition may further comprise a cleaning agent. The cleaning agent may comprise a detergent, an enzyme, or a combination thereof. A non-limiting example of a suitable cleaning agent includes Alconox powder.

A method of sterilizing or sanitizing a device or object is also provided herein. The method comprises applying the nitric oxide precursor described above to the device or object. In embodiments when the nitric oxide precursor is included in the multi-component composition, the method comprises applying the multi-component composition described above to the device or object. As will be appreciated, the step of applying the nitric oxide precursor may also include a step of applying a thiol-containing compound and a nitrosating compound that will react in situ to form the nitric oxide precursor.

A multi-component system for providing nitric oxide is also provided herein. The system comprises a first compartment comprising the first component. As described above, the first component comprises the thiol-containing compound. The system further comprises a second compartment comprising the second component. As also described above, the second component comprises the nitrosating compound. The system further comprises a mixing chamber in fluid communication with the first compartment and the second compartment for combining the first compartment and the second compartment.

### BRIEF DESCRIPTION OF THE DRAWINGS

FIG. 1 is a graph illustrating NO release by a non-limiting embodiment of an exemplary nitric oxide precursor.

FIG. 2 is a graph illustrating NO release by a non-limiting embodiment of another exemplary nitric oxide precursor.

FIG. 3 is a photograph illustrating non-limiting embodiments of exemplary multi-component compositions including nitric oxide precursors.

FIG. 4 is a graph illustrating NO release by non-limiting embodiments of the exemplary multi-component compositions of FIG. 3.

### DETAILED DESCRIPTION

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g., a total weight of dry matter).

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed so as to have the same overall result as if the object were completely enclosed.

The drawings are semi-diagrammatic and not to scale and, particularly, some of the dimensions are for the clarity of presentation and are shown exaggerated in the drawings. Similarly, although the views in the drawings for ease of description generally show similar orientations, this depiction in the drawings is arbitrary. Generally, functionalized polymeric materials can be operated in any orientation. As used herein, it will be understood that when a first element or layer is referred to as being “over,” “overlying,” “under,” or “underlying” a second element or layer, the first element or layer may be directly on the second element or layer, or intervening elements or layers may be present where a straight line can be drawn through and between features in overlying relationship. When a first element or layer is referred to as being “on” a second element or layer, the first element or layer is directly on and in contact with the second element or layer. Further, spatially relative terms, such as “upper,” “over,” “lower,” “under,” and the like, may be used herein for ease of description to describe one element or feature's relationship to another element(s) or feature(s) as illustrated in the figures. It will be understood that the spatially relative terms are intended to encompass different orientations of the functionalized polymeric material in use or operation in addition to the orientation depicted in the figures. For example, if the functionalized polymeric material in the figures is turned over, elements described as being “under” other elements or features would then be oriented “above” the other elements or features. Thus, the exemplary term “under” can encompass either an orientation of above or below. The functionalized polymeric material may be otherwise oriented (rotated 90 degrees or at other orientations) and the spatially relative descriptors used herein may likewise be interpreted accordingly.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following detailed description is merely illustrative in nature and is not intended to limit the embodiments of the subject matter or the application and uses of such embodiments. Furthermore, there is no intention to be bound by any expressed or implied theory presented in the preceding technical field, background, brief summary or the following detailed description.

A nitric oxide precursor for providing nitric oxide is provided herein. The nitric oxide precursor comprises, consists essentially of, consists of, or is, a reaction product of a thiol-containing compound and a nitrosating compound. In preferred embodiments, the thiol-containing compound comprises a primary thiol. The thiol-containing compound and the nitrosating compound are reacted in the presence of a solvent to form the nitric oxide precursor. In some embodiments, the solvent is supplied with the thiol-containing compound and/or the nitrosating compound, or supplied separately, or supplied by air humidity (which can be attracted by a desiccant). As will be appreciated, the reaction can be enhanced by various reducing equivalents such as transition metals, thiols, ascorbic acid, NADH, etc. The so formed nitric oxide precursor is capable of decomposing to form the nitric oxide. An exemplary reaction schematic is shown below:

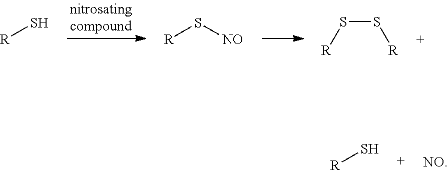

In various embodiments, the nitric oxide precursor comprises a nitrosothiol that is capable of decomposing to form the nitric oxide. It is contemplated therein that the nitric oxide and air will react, resulting in a mixture containing various oxides of nitrogen. Specifically, the addition of nitric oxide to air, or air to nitric oxide, results in the formation of nitric dioxide when nitric oxide reacts with the oxygen in air. The concentration of each nitrogen-oxide species that is present in a mixture may vary with temperature, pressure, and initial concentration of the nitric oxide.

Nitric oxide is lipid soluble and has the ability to disrupt the lipid membranes of microorganisms, to modulate cell and tissue response, to control coagulation and biological integration, to impart antimicrobial characteristics, etc. Furthermore, nitric oxide may inactivate thioproteins thereby disrupting the functional proteins of microbes. Nitrogen dioxide is more water soluble than nitric oxide. Finally, nitric oxide and nitrogen dioxide are effective disruptors of DNA, causing strand breaks and other damage leading to an inability for the cell to function.

As used herein, the term “nitric oxide” or “NO” means the NO free radical. As is well known, NO is chemically instable and readily reacts with oxygen to form various oxides, collectively referred to as NOx. As used herein, the term NOx is an abbreviation for nitrogen oxides or the oxides of nitrogen, which are the oxides formed by nitrogen in which nitrogen exhibits each of its positive oxidation numbers from +1 to +5. As used herein, the terms “nitrogen oxides” and ‘oxides of nitrogen’ and ‘NOx’ mean a gas having one or more of the following gases, all of which contain nitrogen and oxygen in varying amounts: nitric oxide (NO), nitrogen dioxide (NO₂), nitrogen trioxide (NO₃), dinitrogen trioxide (N₂O₃), dinitrogen tetroxide (N₂O₄), dinitrogen pentoxide (N₂O₅) and nitrous oxide (N₂O). As used herein, the phrase “nitric oxide precursor” means a compound or composition capable of producing or releasing NO.

The nitric oxide precursor may be used in a wide variety of medical and consumer applications. The properties of the nitric oxide precursor may be adjusted based on the selection of the thiol-containing compound and the nitrosating compound to suit specific applications. Non-limiting examples of suitable adjustments include nitric oxide generation capabilities and the rate of release of nitric oxide from the nitric oxide precursor. As described in greater detail below, the nitric oxide precursor may be formed in situ from a multi-component composition. This in situ formation of the nitric oxide precursor may also be adjusted to suit specific applications by tuning formation and stability properties of the nitric oxide precursor such that nitric oxide can be, for example, generated rapidly and in a controlled manner. This controlled nitric oxide release is useful for sterilizing and sanitizing medical and consumer devices.

Viewed from a different perspective, the inventors contemplate utilizing the nitric oxide precursor and the multi-component composition to form nitric oxide for a variety of medical and consumer applications. In various embodiments, a device or object may be treated with the multi-component composition, such as in the form of a liquid, a powder, a film, a coating, or the like. Non-limiting examples of suitable uses of the multi-component composition (e.g., as liquids, powders, films, or coatings) include: detergent or cleaning solutions for sanitizing or sterilizing objects treated with the solution (e.g., sports equipment, such as hockey gloves and cycling gloves, cleaning surgical instruments, internal lumens of endoscopes, surfaces of medical equipment, and the like); detergent or cleaning powders for sanitizing or sterilizing objects treated with the powder; hygienic containers for sanitizing hygiene devices (e.g., desiccants and the like); medical device containers for sanitizing medical instruments (e.g., stethoscopes, otoscopes, and the like), medical devices (e.g., portable ultrasound device, communication devices, and the like); components of devices exposed to moisture for resisting growth of mold or mildew (e.g., washing machines, boat compartments, and the like); sporting equipment (e.g., yoga mats, touchable surfaces of strength training equipment, touchable surfaces of cardio equipment, and the like); liners for sporting equipment bags for sanitizing sporting equipment (e.g., shoes, hockey equipment, ski equipment, facemasks, googles, helmets, and the like); food packaging for preserving foodstuff (e.g., meats, fruits, vegetables, cheeses, ingredients thereof, and the like); components of vehicles for sanitizing vehicles (e.g., headliners, seat cushion liners, carpet liners, and the like); and within drawers of cabinets, desks, boxes, etc., to eliminate musty odors. In some or all of the examples above, it should be appreciated that the nitric oxide precursor is preferably not supplied as an already formed compound, but that the nitric oxide precursor is generated in situ by reaction of the thiol-containing compounds with a nitrosating compound in the presence of a solvent (that may be derived from air humidity, supplied separately, or that may be used as a solvent for the thiol-containing compound and/or the nitrosating compound).

Therefore, and as introduced above, a multi-component composition for providing nitric oxide is also provided. The multi-component composition may be utilized for forming solutions that generate nitric oxide in a controlled and predicable manner from a matrix/solution phase. These compositions may comprise a first component comprising the thiol-containing compound and a second component comprising the nitrosating compound to form small molecule nitric oxide donors (preferably, but not necessarily primary nitrosothiols) that can be used for formulations and applications relating to disinfecting, sanitizing, and sterilizing washes for a wide variety of objects and devices and in a number of applications.

In some embodiments, the multi-component composition comprises the first component comprising the thiol-containing compound and the second component comprising the nitrosating compound with the first component and the second component isolated from each other. At least one of the first component and the second component may comprise the solvent. Alternatively, the first and second components are isolated from each other and the solvent is separately provided or first and second components are placed into a solvent. In other embodiments, the multi-component composition comprises the first component comprising the thiol-containing compound having a particle size of from about 1 nm to about 10 mm, alternatively from about 1 nm to about 1 mm, alternatively from about 1 nm to about 500 μm, or alternatively from about 10 nm to about 500 μm. Likewise, the multi-component composition further comprises the second component comprising the nitrosating compound having a particle size of from about 1 nm to about 10 mm, alternatively from about 1 nm to about 1 mm, alternatively from about 1 nm to about 500 μm, or alternatively from about 10 nm to about 500 μm. Regardless of the arrangement, it should be once more appreciated that the nitric oxide precursor will be formed in situ, which is then subject to decomposition to form nitric oxide.

With respect to the multi-component composition, the inventors contemplate that the nitric oxide precursor may exhibit decomposition to nitric oxide after forming the reaction product within a predetermined amount of time, such as within 1 hour, alternatively within 30 minutes, alternatively within 5 minutes, alternatively within 1 minute, or alternatively within 10 seconds, alternatively within 1 second, or alternatively within 0.1 seconds. Viewed from a different perspective, the multi-component composition may exhibit decomposition to nitric oxide after forming the nitric oxide precursor within a predetermined amount of time, such as from about 0.01 seconds to about 1 hour, alternatively from about 0.01 seconds to about 30 minutes, alternatively from about 1 second to about 5 minutes, or alternatively from about 1 second to about 1 minute. In various embodiments, decomposition to nitric oxide may be sustained for a predetermined amount of time, such as a time period of from about 1 minutes to about 1 year, alternatively from about 1 hour to about 6 months, alternatively from about 24 hours to about 3 months, or alternatively from about 1 week to about 8 weeks. From a different perspective, decomposition to nitric oxide may be sustained for a period of at least 1 minute, alternatively at least 1 hours, alternatively at least 24 hours. or alternatively at least 1 week. Without being bound by theory, it is believed that in situ formation of the reaction product provides controlled decomposition of the nitric oxide precursor (e.g., nitrosothiol) to nitric oxide within the predetermined amount of time for the predetermined amount of time.

As will be readily appreciated, decomposition rate of the nitric oxide precursor will depend on a variety of factors which can be fine-tuned to achieve a desired decomposition profile. Among other factors, the decomposition rate can be modified by selecting the type of nitrosothiol, the molar ratios of the thiol-containing compound and the nitrosating compound, the presence/quantity of reduction equivalents or other catalysts that influence the in situ formation of the nitric oxide precursor, the pH of the solution, and availability of solvent. For example, it should be appreciated that primary nitrosothiols have a substantially higher rate of decomposition than secondary or tertiary nitrosothiols. Moreover, in situ formation of the nitrosothiols may be accelerated in the presence of various reduction equivalents such as thiols, NADH, transition metals, ascorbic acid, etc. (such reducing agents could be present in trace amounts to equimolar ratio, or even be present in molar excess relative to the thiol-containing agent). Likewise, acidic pH (e.g., pH<4.0) will generally favor stability of nitrosothiols whereas basic pH (e.g., pH>7.0) will favor decomposition of the nitrosothiol. Still further, where the solvent is water from humid air, the degree of humidity will determine the rate of the in situ formation of the nitric oxide precursor.

Regardless of the reaction conditions, it is generally contemplated that in some embodiments the rate of decomposition of the nitric oxide precursor to form nitric oxide will be greater than the rate of in situ formation such as 1.5 time greater, or 2.5 times greater, or 5 times greater or ten times greater, or even more. In other embodiments, the rate of decomposition of the nitric oxide precursor to form nitric oxide will be lesser than the rate of in situ formation such as 1.5 time less, or 2.5 times less, or 5 times less or ten times less, or even less. Moreover, and depending on the particular formulation and nitric oxide precursor, it is contemplated that in some embodiments the nitric oxide released within 1 hour will be at least 25%, or at least 50%, or at least 70%, or at least 80%, or at least 90%, or at least 95% of all releasable nitric oxide in the formulation. In further embodiments, it is contemplated that the nitric oxide released within 24 hours will be at least 25%, or at least 50%, or at least 70%, or at least 80%, or at least 90%, or at least 95% of all releasable nitric oxide in the formulation, and in still other embodiments, it is contemplated that the nitric oxide released within 7 days will be at least 25%, or at least 50%, or at least 70%, or at least 80%, or at least 90%, or at least 95% of all releasable nitric oxide in the formulation. In additional embodiments, it is contemplated that the nitric oxide released within 1 month will be at least 25%, or at least 50%, or at least 70%, or at least 80%, or at least 90%, or at least 95% of all releasable nitric oxide in the formulation.

The inventors contemplate that small molecular nitric oxide precursors can be blended into polymers, solution phases, or powders and used to generate nitric oxide for creating a wide array of NO donating/generating entities by blending the nitric oxide precursor into the matrix (polymer or powder or embedded in and on solid carriers) or for creating solutions that generate NO in a controlled and predicable manner from the matrix/solution phase. Herein we describe a method for synthesizing novel small molecule NO donors that can be used for this purpose and formulations and applications of these NO generating moieties that can be used as disinfecting, sanitizing, and sterilizing washes for a wide array of different objects and in a number of different situations.

Nitric oxide precursors, such as small molecule nitrosothiols (and especially primary nitrosothiols), formed in situ from thiol-containing compounds (e.g., cysteine, N-acetylcysteine, and glutathione) and nitrosating compound (e.g., organo-nitrite or inorganic nitrite salt) may be formed in both organic and aqueous phases. Solutions that contain cleaning agents (e.g., enzymes, surfactants, etc.) may be combined with the specific thiol-containing compound and an appropriate nitrosating compound under controlled conditions to form the nitrosothiol. This nitrosothiol can then decompose rapidly to generate nitric oxide. These 2-part systems (thiol-containing compound and nitrosating compound) in combination with various carriers and cleaning agents, such as powders or solution components (e.g., silica gel, sodium polyacrylate, Alconox soap, proteases, etc.) may be used for reprocessing endoscopes and probes and sterilizing other objects.

Non-limiting examples of suitable embodiments include silica gel desiccant combined with cysteine and sodium nitrite as a dry powder that can generate nitric oxide as the mixture adsorbs moisture from humid air. Another example is combining the thiol-containing compound and a sodium nitrite solution together and pouring this solution into objects, such as shoes, to eliminate odors resulting from bacteria.

Referring back to the thiol-containing compound utilized to form the reaction product, there is a wide variety of possible thiol-containing compounds that can be used, depending on the design constraints of the desired application of the multi-component composition. Among other choices, primary thiols are typically preferred. The thiol-containing compound can include, but is not limited to, one or more of 1,2-ethane dithiol, 2,3-dimercaptopropanol, pyrithione, dithioerythritol, 3,4-dimercaptotoluene, 2,3-butanedithiol, 1,3-propanedithiol, 2-hydroxypropane thiol, 1-mercapto-2-propanol, dithioerythritol and dithiothreitol. Other exemplary thiol-containing compounds include alpha-lipoic acid, methanethiol (CH₃SH [m-mercaptan]), ethanethiol (C₂H₅SH [e-mercaptan]), 1-propanethiol (C₃H₇SH [n-P mercaptan]), 2-propanethiol (CH₃CH(SH)CH₃ [2C3 mercaptan]), butanethiol (C₄H₉SH ([n-butyl mercaptan]), tert-butyl mercaptan (C(CH₃)₃SH [t-butyl mercaptan]), pentanethiols (C₅H₁₁SH [pentyl mercaptan]), coenzyme A, lipoamide, glutathione, cysteine, cystine, 2-mercaptoethanol, dithiothreitol, dithioerythritol, 2-mercaptoindole, transglutaminase, (11-mercaptoundecyl) hexa (ethylene glycol), (11-mercapto-undecyl)tetra(ethylene glycol), (11-mercaptoundecyl)tetra(ethylene glycol) functionalized gold nanoparticles, 1,1′,4′,1″-terphenyl-4-thiol, 1,11-undecanedithiol, 1,16-hexadecanedithiol, 1,2-ethanedithiol, 1,3-propanedithiol, 1,4-benzenedimethanethiol, 1,4-butanedithiol, 1,4-butane-dithiol diacetate, 1,5-pentanedithiol, 1,6-hexanedithiol, 1,8-octanedithiol, 1,9-nonanedithiol, adamantanethiol, 1-butanethiol, 1-decanethiol, 1-dodecanethiol, 1-heptanethiol, 1-heptanethiol, 1-hexadecanethiol, 1-hexanethiol, 1-mercapto-(triethylene glycol), 1-mercapto-(triethylene glycol) methyl ether functionalized gold nanoparticles, 1-mercapto-2-propanol, 1-nonanethiol, 1-octadecanethiol, 1-octanethiol, 1-octanethiol, 1-pentadecanethiol, 1-pentanethiol, 1-propanethiol, 1-tetradecanethiol, 1-undecanethiol, 11-(1H-pyrrol-1-yl) undecane-1-thiol, 11-amino-1-undecane-thiol hydrochloride, 11-bromo-1-undecanethiol, 11-mercapto-1-undecanol, 11-mercapto-1-undecanol, 11-mercaptoundecanoic acid, 11-mercaptoundecanoic acid, 11-mercaptoundecyl trifluoroacetate, 11-mercaptoundecylphosphoric acid, 12-mercaptododecanoic acid, 12-mercaptododecanoic acid, 15-mercaptopentadecanoic acid, 16-mercaptohexadecanoic acid, 16-mercaptohexadecanoic acid, 1H, 1H,2H,2H-perfluorodecanethiol, 2,2′-(ethylenedioxy)di-ethanethiol, 2,3-butanedithiol, 2-butanethiol, 2-ethylhexanethiol, 2-methyl-1-propanethiol, 2-methyl-2-propanethiol, 2-phenylethanethiol, 3,3,4,4,5,5,6,6,6-nonafluoro-1-hexanethiol purum, 3-(dimethoxymethylsilyl)-1-propanethiol, 3-chloro-1-propanethiol, 3-mercapto-1-propanol, 3-mercapto-2-butanol, 3-mercapto-N-nonylpropionamide, 3-mercaptopropionic acid, 3-mercaptopropyl-functionalized silica gel, 3-methyl-1-butanethiol, 4,4′-bis(mercapto-methyl) biphenyl, 4,4′-dimercaptostilbene, 4-(6-mercaptohexyloxy)benzyl alcohol, 4-cyano-1-butanethiol, 4-mercapto-1-butanol, 6-(ferrocenyl) hexanethiol, 6-mercapto-1-hexanol, 6-mercaptohexanoic acid, 8-mercapto-1-octanol, 8-mercaptooctanoic acid, 9-mercapto-1-nonanol, biphenyl-4,4′-dithiol, butyl 3-mercaptopropionate, copper (I) 1-butanethiolate, cyclohexanethiol, cyclopentanethiol, decanethiol functionalized silver nanoparticles, dodecanethiol functionalized gold nanoparticles, dodecanethiol functionalized silver nanoparticles, hexa (ethylene glycol) mono-11-(acetylthio) undecyl ether, mercaptosuccinic acid, methyl 3-mercaptopropionate, octanethiol functionalized gold nanoparticles, PEG dithiol, S-(11-bromoundecyl)thioacetate, S-(4-cyanobutyl)thioacetate, thiophenol, triethylene glycol mono-11-mercaptoundecyl ether, trimethylolpropane tris(3-mercaptopropionate), [11-(methylcarbonylthio) undecyl]tetra(ethylene glycol), m-carborane-9-thiol, p-terphenyl-4,4″-dithiol, tert-dodecylmercaptan, or tert-nonyl mercaptan.

In certain embodiments, the thiol-containing compound includes a cysteine or derivative thereof, a thiol-derivatized polymer or filler, or a combination thereof. In embodiments when the cysteine or derivative thereof is utilized, the cysteine or derivative thereof may comprise cysteine, glutathione, acetyl cysteine, penicillamine, acetylpenicillamine, S-nitroso-n-acetylpenicillamine, bucillamine, or combinations thereof. It is to be appreciated that the thiol-containing compound may be included as part of a peptide or other macromolecules so long as the thiol-containing compound is compatible with the components of the multi-component composition. In embodiments when the cysteine or derivative thereof is utilized as part of a peptide, the peptide may include any combination of amino acids so long as the peptide includes the cysteine or derivative thereof as at least one of the constituents of the peptide. Non-limiting examples of suitable cysteines or derivatives thereof are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety.

In various embodiments, the thiol-containing compound has a weight average molecular weight of no greater than 500,000 g/mol, alternatively no greater than 100,000 g/mol, alternatively no greater than 10,000 g/mol, alternatively no greater than 1,000 g/mol, or alternatively no greater than 500 g/mol. Viewed from a different perspective, the thiol-containing compound may have a weight average molecular weight of from about 10 g/mol to about 500,000 g/mol, alternatively from about 10 g/mol to about 100,000 g/mol, alternatively from about 10 g/mol to about 1,000 g/mol, or alternatively from about 10 g/mol to about 500 g/mol. For example, cysteine has a weight average molecular weight of 121 g/mol, glutathione has a weight average molecular weight of 307.33 g/mol, butylthiol has a weight average molecular weight of 90.19 g/mol, and serum albumin has a weight average molecular weight of 66 kDa. Without being bound by theory, it is believed that reaction kinetics of forming the reaction product is improved by utilizing the thiol-containing compounds having lower weight average molecular weights. This improved reaction kinetics provides rapid generation of nitric oxide resulting from the in situ formation of the reaction product. Moreover, primary nitrosothiol compounds will generally more rapidly decompose to form nitric oxide than secondary or tertiary nitrosothiols.

Referring back to the nitrosating compound utilized to form the reaction product, the nitrosating compound may be any compound serving as a source of nitroso groups and will in general be a compound of formula NOX where X is an organic or inorganic anion or a group OR₂ where R₂ is an organic group. X may thus be an organic anion derived from a carboxylic acid, e.g., an alkane carboxylic acid containing 2-7 carbon atoms, nitrosating agents of this type including acetyl nitrite and propionyl nitrite. Where X is an inorganic anion this may be derived from, for example, a mineral acid, e.g., a halide ion such as chloride or bromide or a sulphate ion, or from a Lewis acid, e.g., a borofluoride ion. Other inorganic anions include hydroxide and sulphonate. Nitrosating compounds of this type thus include nitrosyl chloride, nitrosyl sulphate, nitrosyl borofluoride, nitrous acid and Fremys salt (potassium nitrosyldisulphonate). Where X is a group of formula OR2 the organic group R2 may be, for example, a lower alkyl group, such as containing 1-9 carbon atoms, e.g., ethyl, n-propyl, isopropyl, n-butyl, t-butyl or isopentyl.

In certain embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, nitrite salts, ion paired nitrite, silver nitrite, zinc nitrite, iron nitrite, copper nitrite, transition metal-nitrite compounds, or combinations thereof. Also, nitric oxide gas may be used as a nitrosating agent.

In various embodiments, the nitrosating compound has a weight average molecular weight of no greater than 10,000 g/mol, alternatively no greater than 1,000 g/mol, alternatively no greater than 500 g/mol, or alternatively no greater than 250 g/mol. From a different perspective, the nitrosating compound may have a weight average molecular weight of from about 10 g/mol to about 10,000 g/mol, alternatively from about 10 g/mol to about 1,000 g/mol, alternatively from about 10 g/mol to about 500 g/mol, or alternatively from about 10 g/mol to about 250 g/mol. For example, NaNO2 has a weight average molecular weight of 69 g/mol and butyl nitrite has a weight average molecular weight of 103 g/mol. Without being bound by theory, it is believed that reaction kinetics of forming the reaction product is improved by utilizing the nitrosating compounds having lower weight average molecular weights. As described above, this improved reaction kinetics provides rapid generation of nitric oxide resulting from the in situ formation of the reaction product.

As introduced above, the thiol-containing compound and the nitrosating compound may be reacted in the presence of the solvent to form the reaction product (e.g., (primary) nitrosothiol). If utilized, the solvent may be included in various amounts. The solvent may be aqueous, organic, non-organic, or combinations thereof. In certain embodiments, the solvent is an aqueous solvent, such as water or a mixture of water and methanol. In other embodiments, the solvent may comprise an organic solvent, such as tetrahydrofuran. Other non-limiting examples of suitable solvents include aromatics, aliphatics, ketones, such as methyl ethyl ketone, isobutyl ketone, ethyl amyl ketone, acetone, alcohols, such as methanol, ethanol n-butanol isopropanol esters, such as ethyl acetate, glycols, such as ethylene glycol propylene glycol ethers, such as tetrahydrofuran, ethylene glycol mono butyl ether, or combinations thereof.

In various embodiments, the thiol-containing compound and the nitrosating compound is reacted in the presence of an acid. The acid may be utilized to improve formation and/or stability of the reaction product, such as when utilizing thiol-containing compounds comprising cysteine. In certain embodiments, the acid may comprise hydrochloric acid. Other non-limiting examples of suitable acids includes citric acid, methane sulfonic acid, ethane sulfonic acid, propane sulfonic acid, butane sulfonic acid, acetic acid, hydroxy acetic acid, propionic acid, hydroxy propionic acid, a-ketopropionic acid, butyric acid, mandelic acid, valeric acid, succinic acid, tartaric acid, malic acid, oxalic acid, fumaric acid, adipic acid, maleic acid, sorbic acid, benzoic acid, succinic acid, glutaric acid, adipic acid, α-hydroxy acids, ethylenediaminetetraacctic acid (EDTA), phosphonic acid, octyl phosphoric acid, acrylic acid, polyacrylic acid, aspartic acid, polyaspartic acid, p-hydroxybenzoic acids, iminoacetic acids, or combinations thereof. It is to be appreciated that the acid may be included as part of any component of the composition (e.g., carrier, solvent, etc.) or the reactants of the reaction product.

In other embodiments, the reaction product is formed substantially free of the presence of an acid, such as when utilizing thiol-containing compounds comprising glutathione. The inventors contemplate that the multi-component composition substantially free of the acid exhibits improved compatibility with the device or object. The phrase “substantially free” as used herein refers to either the complete absence of the acid or a minimal amount thereof merely as impurity, unintended byproduct of another ingredient, or in an amount that has a negligible impact on the composition. In certain embodiments, “substantially free” means that the acid is present in the composition in an amount of less than 0.5 wt. %, less than 0.25 wt. %, less than 0.1 wt. %, less than 0.05 wt. %, or less than 0.01 wt. %, or even 0 wt. %, based on a total weight of the composition.

In still other embodiments, the reaction product is formed in the presence of a reducing equivalent, and suitable reducing equivalents include various metals and transition metals, dithionates, thiosulfates, hydrazine, oxalic acid, ascorbic acid, formic acid, NADH, NADPH, etc. As will be readily recognized, these reducing equivalents can be separately provided, can be provided within the solvent, or admixed with the thiol-containing compound and/or the nitrosating compound.

In these and other embodiments, the multi-component composition further comprises a carrier. The carrier may comprise a silica gel, a sodium polyacrylate, or a combination thereof. However, it is to be appreciated that any other carrier may be utilized. Such carriers are well known to those of skill in the art and are described in textbooks such as Remington's Pharmaceutical Sciences, Mack Publishing Co., Easton, Pa., 1985, which is incorporated by reference in its entirety.

The multi-component composition may comprise the thiol-containing compound in an amount of from about 1 to about 80 wt. %, alternatively from about 1 to about 70 wt. %, or alternatively from about 50 to about 80 wt. %, based on a total weight of the composition. The multi-component composition may comprise the nitrosating compound in an amount of from about 1 to about 75 wt. %, alternatively from about 1 to about 10 wt. %, or alternatively from about 10 to about 75 wt. %, based on a total weight of the composition. The multi-component composition may comprise the carrier in an amount of from about 10 to about 95 wt. %, alternatively from about 10 to about 80 wt. %, or alternatively from about 80 to about 95 wt. %, based on a total weight of the composition. The multi-component composition may comprise the solvent in an amount of from about 1 to about 99 wt. % based on a total weight of the composition.

In exemplary embodiments, the multi-component composition may be formulated as a detergent composition or a cleaning composition. As used herein the phrases “detergent composition” or “cleaning composition” includes compositions and formulations designed for cleaning soiled material. Such compositions include, but are not limited to, object cleaning composition, medical device cleaning compositions, hard surface cleaning compositions, dishware cleaning compositions, laundry cleaning compositions and detergents, spray products, dry cleaning agent or composition, unit dose formulation, delayed delivery formulation, detergent contained on or in a porous substrate or nonwoven sheet, detergent contained on or in a water-soluble film, and other suitable forms that may be apparent to one skilled in the art in view of the teachings herein. The multi-component composition may have a form selected from liquid, powder, single-phase or multi-phase unit dose, 2 layer paper carrier, pouch, tablet, gel, paste, bar, or flake.

In these and other embodiments, the detergent or cleaning composition further comprises a cleaning agent. The cleaning agent comprises a detergent, an enzyme, or a combination thereof. A non-limiting example of a suitable cleaning agent include Alconox powder. In embodiments when the cleaning agent comprises a detergent, the detergent may comprise a surfactant, such as amine oxide, alkylbenzene sulfonate, alkyl ether sulfate, fatty alcohol ethoxylate, alkyl glycosides, alkoxylated fatty acid alkyl esters, amine oxides, fatty acid alkanolamides, hydroxy mixed ethers, sorbitan fatty acid esters, polyhydroxy fatty acid amides, and alkoxylated alcohols.

In embodiments when the cleaning agent comprises an enzyme, the enzyme may comprise one or more enzymes, which can display a catalytic activity in a detergent, such as a protease, amylase, lipase, cellulase, hemicellulase, mannanase, pectin-cleaving enzyme, tannase, xylanase, xanthanase, β-glucosidase, carrageenase, perhydrolase, oxidase, oxidoreductase, and mixtures thereof. In certain embodiments, the enzyme comprises proteases, amylases (e.g., α-amylases), cellulases, lipases, hemicellulases, pectinases, mannanases, β-glucanases, or combinations thereof. The properties of the enzyme should be compatible with the multi-component composition (i.e. pH-optimum, compatibility with other enzymatic and non-enzymatic ingredients, etc.). If utilized, the enzyme should be present in effective amounts.

When utilized, the protease may be of animal, vegetable or microbial origin, including chemically or genetically modified mutants. Microbial origin is preferred. It may be an alkaline protease, such as a serine protease or a metalloprotease. A serine protease may for example be of the Si family, such as trypsin, or the S8 family such as subtilisin. A metalloproteases protease may for example be a thermolysin from e.g., family M4, M5, M7 or M8.

When utilized, suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g. H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131:253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO2007/087508 and WO 2009/109500, which are incorporated by reference in their entirety.

When utilized, suitable amylases include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839. Examples of useful amylases are the variants described in WO 94/02597, WO 94/18314, WO 96/23873, and WO 97/43424, which are incorporated by reference in their entirety.

When utilized, suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757, and WO 89/09259, which are incorporated by reference in their entirety.

When utilized, suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257, which are incorporated by reference in their entirety.

The detergent or cleaning composition may further include additives, such as builders, bleaching agents, electrolytes, nonaqueous solvents, pH adjusting agents, fragrances, perfume carriers, fluorescent agents, dyes, hydrotropes, foam inhibitors, silicone oils, anti-redeposition agents, graying inhibitors, shrinkage preventers, anti-creasing agents, dye transfer inhibitors, antimicrobial substances, germicides, fungicides, antioxidants, preservatives, corrosion inhibitors, antistatic agents, bitter agents, ironing aids, hydrophobizing and impregnating agents, swelling and anti-slip agents, softening components, and UV absorbers.

The detergent or cleaning composition may comprise the thiol-containing compound in an amount of from about 0.01 to about 40 wt. %, alternatively from about 0.01 to about 5 wt. %, alternatively from about 5 to about 10 wt. %, or alternatively from about 10 to about 40 wt. %, based on a total weight of the composition. The detergent or cleaning composition may comprise the nitrosating compound in an amount of from about 0.1 to about 40 wt. % or alternatively from about 0.1 to about 10 wt. %, based on a total weight of the composition. The detergent composition or a cleaning composition may comprise the cleaning agent in an amount of from about 1 to about 99 wt. % based on a total weight of the composition. The detergent composition or a cleaning composition may comprise the solvent in an amount of from about 1 to about 99 wt. % based on a total weight of the composition.

In various embodiments, the detergent or cleaning composition described herein can be filled into a water-soluble envelope and thus be part of a water-soluble package. The water-soluble envelope may be formed by a water-soluble film material. Such water-soluble packages can be produced either by vertical form fill seal (VFFS) methods or by thermoforming methods.

The envelope can be made of one layer or of two or more layers of the water-soluble film material. The water-soluble film material of the first layer and of the other layers, if present, can be the same or different. The water-soluble envelope, for example, is made from a water-soluble film material selected from the group comprising polymers or polymer mixtures. The water-soluble envelope may contain polyvinyl alcohol or a polyvinyl alcohol copolymer. Polymers selected from the group comprising acrylic acid-containing polymers, polyacrylamides, oxazoline polymers, polystyrene sulfonates, polyurethanes, polyesters, polyether polylactic acid, and/or mixtures of the above polymers, can be added to a film material that is suitable for producing the water-soluble envelope.

The thermoforming method generally includes forming a first layer from a water-soluble film material to create convexities for receiving a composition therein, filling the composition into the convexities, covering the convexities filled with the composition with a second layer of a water-soluble film material, and sealing the first and second layers together at least around the convexities. The water-soluble package comprising the liquid washing and the water-soluble envelope can have one or more chambers. The water-soluble packages can have a substantially dimensionally stable spherical and pillow-shaped configuration with a circular, elliptical, square, or rectangular basic form. The chambers may be isolated from one another.

A method of sterilizing or sanitizing a device or object is also provided herein. The method comprises applying the nitric oxide precursor described above to the device or object. In embodiments when the nitric oxide precursor is included in the multi-component composition, the method comprises applying the multi-component composition described above to the device or object.

A multi-component system for providing nitric oxide is also provided herein. The system comprises a first compartment comprising the first component. As described above, the first component comprises the thiol-containing compound. The system further comprises a second compartment comprising the second component. As also described above, the second component comprises the nitrosating compound. The system further comprises a mixing chamber in fluid communication with the first compartment and the second compartment for combining the first compartment and the second compartment.

A method of forming the nitric oxide precursor is provided herein. The method comprises combining the thiol-containing compound and the nitrosating compound in the presence of a solvent to form the nitric oxide precursor.

In one exemplary embodiment, the step of combining comprises (a) combining the thiol-containing compound a solvent to form a first solution, (b) combining the nitrosating compound and a solvent to form a second solution, and (c) combining the first solution and the second solution to form the nitric oxide precursor.

In another exemplary embodiment, the step of combining comprises (a) combining the thiol-containing compound, a solvent, and a cleaning agent to form a first solution and (b) combining the nitrosating compound and the first solution to form the nitric oxide precursor. In these and other embodiments, when an acid is utilized, the acid may be combined with the first solution as well.

In yet another embodiment, step of combining comprises (a) combining the thiol-containing compound, the nitrosating compound, and the carrier, with each in particle form (i.e., substantially free of a solvent) to form a first mixture, and (b) combining a solvent and the first mixture to form the nitric oxide precursor.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1: Exemplary GNSO Desiccant Powder

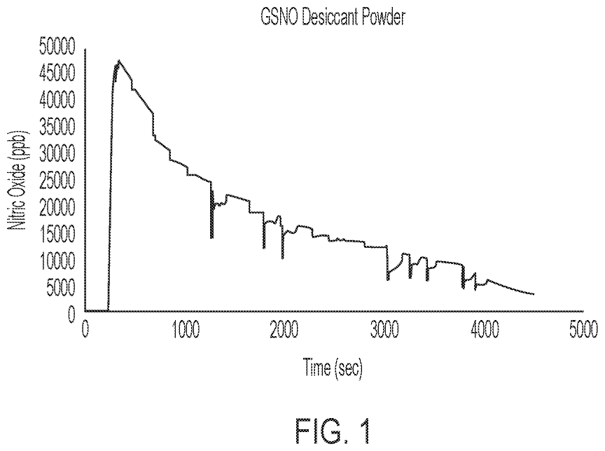

100 mg reduced glutathione, 150 mg NaNO2, and 2.5 g sodium polyacrylate were mixed as a dry powder. When exposed to water vapor, NO was generated. When the mixture dries (i.e. removal of water vapor and time for hydrophilic polymer to dry, NO release stops. When water vapor is reintroduced, NO is again generated. Nitric oxide formation is shown in FIG. 1.

### Example 2: Exemplary NOCys Desiccant Powder

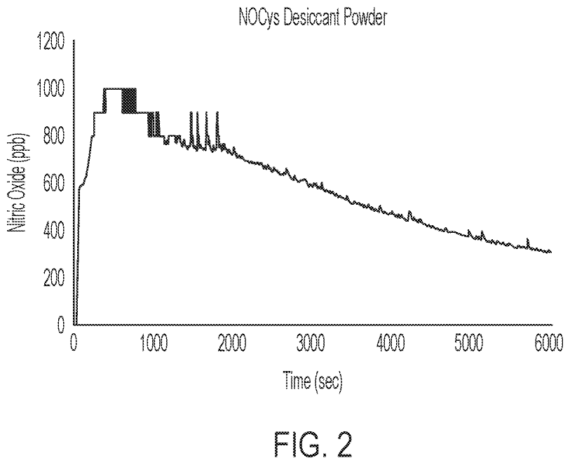

100 mg cysteine, 300 mg NaNO2, and 2 g of sodium polyacrylate were mixed as a dry powder. Approximately 125 mg of this mixture was placed in Tyvek envelop and heat sealed. The packet was exposed to water vapor and released NO. Nitric oxide formation is shown in FIG. 2.

### Example 3: Exemplary GSNO and Alconox Detergent

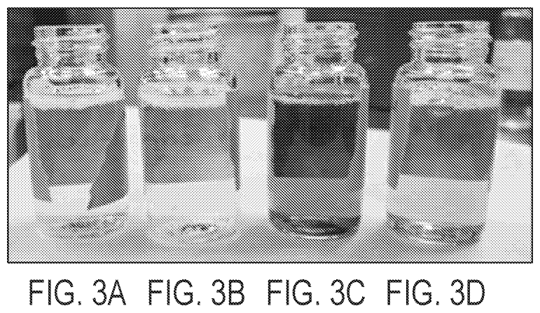

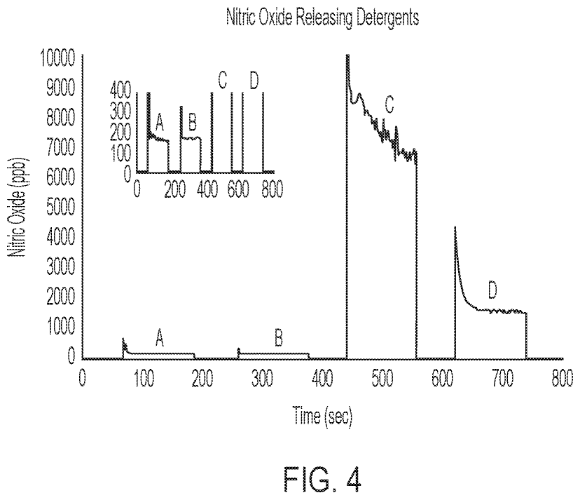

100 mg glutathione was dissolved in 4 mL water with 130 mg of Alconox powder. 34.5 mg of NaNO2 and 100 μL 1M HCl were added to the solution. This soap released NO. See A of FIGS. 3 and 4.

### Example 4: Exemplary NOCys and Alconox Detergent

50 mg Cysteine was dissolved in 4 mL water with 130 mg of Alconox powder. 34.5 mg of NaNO2 and 100 μL 1M HCl were added to the solution. This soap released NO. See B of FIGS. 3 and 4.

### Example 5: Exemplary GSNO Protease Detergent

100 mg Glutathione was dissolved in 4 mL water. 34.5 mg of NaNO2 was added to the solution. This detergent instantly turned deep ruby red and releases NO. See C of FIGS. 3 and 4.

### Example 6: Exemplary NOCys Protease Detergent

50 mg Cysteine was dissolved in 4 mL water. 34.5 mg of NaNO2 and 100 μL 1M HCl were added to the solution. This detergent turned red-pink releases NO. See D of FIGS. 3 and 4.

### Example 7: Exemplary GSNO Two-component Solution

0.01M Glutathione solution was made in water. 0.1M NaNO2 solution was made in water. The 5 mL of each solution were combined and turn bright red. The solution was poured into odor-offensive athletic shoes and tested after 12 hours. No odor remained after 12 hours in the shoes.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A method of sterilizing or sanitizing an article with nitric oxide, comprising:
contacting the article with a reaction mixture comprising a solvent, a thiol-containing compound, and a nitrosating compound;
wherein the thiol-containing compound and the nitrosating compound react in situ on or near the article to form a nitrosothiol compound;
wherein the nitrosothiol compound decomposes to form nitric oxide while the article is in contact with the reaction mixture; and
wherein the nitric oxide is formed in an amount that sterilizes or sanitizes the article.

2. The method of claim 1, wherein the nitrosothiol compound comprises a primary nitrosothiol.

3. The method of claim 1, wherein the nitrosothiol compound decomposes to nitric oxide within 1 hour after forming the nitric oxide precursor.

4. The method of claim 1, wherein the thiol-containing compound comprises a cysteine or derivative thereof, a thiol-derivatized polymer or filler, or a combination thereof.

5. The method of claim 1, wherein the thiol-containing compound comprises a cysteine or derivative thereof.

6. The method of claim 5, wherein the cysteine or derivative thereof comprises cysteine, glutathione, acetyl cysteine, penicillamine, acetylpenicillamine, S-nitroso-n-acetylpenicillamine, bucillamine, or combinations thereof.

7. The method of claim 1, wherein the nitrosating compound comprises a nitrite.

8. The method of claim 7, wherein the nitrite comprises sodium nitrite, calcium nitrite, potassium nitrite, tetrabutylammonium nitrite, dicyclohexylammonium nitrite, butylnitrite, isobutylnitrite, t-butylnitrite, amylnitrite, pentylnittrite, nitrite salts, ion paired nitrite, silver nitrite, zinc nitrite, iron nitrite, copper nitrite, transition metal-nitrite compounds, or combinations thereof.

9. The method of claim 1, wherein the nitrosothiol compound is formed in the presence of an acid.

10. The method of claim 9, wherein the acid comprises hydrochloric acid, citric acid, methane sulfonic acid, ethane sulfonic acid, propane sulfonic acid, butane sulfonic acid, acetic acid, hydroxy acetic acid, propionic acid, hydroxy propionic acid, α-ketopropionic acid, butyric acid, mandelic acid, valeric acid, succinic acid, tartaric acid, malic acid, oxalic acid, fumaric acid, adipic acid, maleic acid, sorbic acid, benzoic acid, succinic acid, glutaric acid, adipic acid, α-hydroxy acids, ethylenediaminetetraacctic acid (EDTA), phosphonic acid, octyl phosphoric acid, acrylic acid, polyacrylic acid, aspartic acid, polyaspartic acid, p-hydroxybenzoic acids, iminoacetic acids, or combinations thereof.

11. The method of claim 9, wherein the thiol-containing compound comprises cysteine.

12. The method of claim 1, wherein the nitrosothiol compound is formed in the absence of an acid.

13. The method of claim 12, wherein the thiol-containing compound comprises glutathione.

14. The method of claim 1, wherein the reaction mixture is formulated as a cleaning composition or a desiccant composition in which the solvent is provided by air humidity.

15. A multi-component composition for providing nitric oxide, the multi-component composition comprising:
a first component comprising a thiol-containing compound;
a second component comprising a nitrosating compound;
wherein the first component and the second component are isolated from each other;
wherein at least one of the first component and the second component further comprises a solvent; and
wherein the thiol-containing compound and the nitrosating compound have a composition such that the thiol-containing compound and the nitrosating compound react, in the presence of the solvent, to form a nitric oxide precursor that decomposes to form nitric oxide.

16. The multi-component composition of claim 15, wherein at least one of the first component and the second component further comprises a carrier or a cleaning agent.

17-19. (canceled)

20. The multi-component composition of claim 15, wherein the nitric oxide precursor comprises a primary nitrosothiol, and/or wherein the nitric oxide precursor decomposes to form the nitric oxide within 1 hour after forming the nitric oxide precursor.

21. (canceled)

22. The multi-component composition of claim 15, wherein the thiol-containing compound comprises a cysteine or derivative thereof, a thiol-derivatized polymer or filler, or a combination thereof.

23-24. (canceled)

25. The multi-component composition of claim 15, wherein the nitrosating compound comprises a nitrite.

26. (canceled)

27. The multi-component composition of claim 15 further comprising an acid.

28-52. (canceled)
